(19) United States
(12) Patent Application Publication
(10) Pub. No.: US 2003/0204186 A1
(43) Pub. Date: Oct. 30, 2003

(54) Ablation device for cardiac tissue, in particular for forming linear lesions between two vessel orifices in the heart

(75) Inventor: Wolfgang Geistert, Rheinfelden (DE)

Correspondence Address: BROWDY AND NEIMARK, P.L.L.C., 624 NINTH STREET, NW, SUITE 300, WASHINGTON, DC, 20001-5303, US

(73) Assignee: Biotronik Mess-und Therapiegerate GmbH & Co. Ingen, Berin (DE)

(21) Appl. No.: 10/419,919

(22) Filed: Apr. 22, 2003

(30) Foreign Application Priority Data
Apr. 24, 2002 (DE) 102 18 426.7

(51) Int. Cl. A61B 18/18

(52) U.S. Cl. 606/41

(57) ABSTRACT

An ablation device for cardiac tissue, in particular for forming linear lesions between two vessel orifices in the heart, comprising

an ablation catheter which, in front of a distal end, is provided with an ablation applicator that reaches over a certain length of the catheter;

a steerable positioning catheter which the ablation catheter is guided along for displacement relative thereto in a longitudinal axial direction; and

a distal end section of the positioning catheter, proximally of which the guide of the ablation catheter through the positioning catheter terminates and the ablation catheter is freely movable together with the ablation applicator, with the effective ablation length, outside the guide, of the ablation applicator being variable by longitudinal axial displacement of the two catheters relative to each other.

### BACKGROUND OF THE INVENTION

1. Field of the Invention

The invention relates to an ablation device for cardiac tissue, in particular for forming linear lesions between two vessel orifices in the heart, comprising an ablation catheter which, in front of a distal end, is provided with an ablation applicator that reaches over a certain length of the catheter;.

2. Background Art

Regarding the background of the invention it can be stated that catheter ablation is a therapy that is used to an increasing degree to treat certain types of arrhythmia. In the process, a lesion—i.e., a denaturation of tissue along the line of a tissue scarring—is created with the aid of the ablation applicator of the catheter at a certain location in the myocardial tissue in order to sever the faulty electrical stimulus pathways at that location that are responsible for arrhythmia. The introduction of energy into the myocardial tissue via the ablation applicator, as a rule, takes place by means of ablation electrodes that operate with high-frequency current. Other forms of energy, such as microwave energies, high-energy direct current or, in principle, other denaturing mechanisms, such as freezing or chemicals (for example alcohol), may also be used for the ablation. The term “ablation applicator”, as it is used in the present application also in connection with the subject matter of the invention, shall always mean all of the listed ablation options, with ablation electrodes representing the most common variant.

For special treatment of so-called atrial fibrillation, it is necessary to connect the orifice openings of the pulmonary veins into the left atrium by linear lesions. This is difficult to achieve with conventional ablation catheters, the desired positions of ablation being hard to reach and keep stable.

To cope with these problems, WO 98/49957 A1 and U.S. Pat. No. 6,164,283 A devise a special ablation arrangement that corresponds to the preamble of claim 1. This ablation device comprises a steerable ablation catheter which, in front of its distal end, is provided with an ablation applicator that extends over a certain length of the catheter. In this active part, the ablation catheter is positioned and kept stable in the vicinity of the pulmonary vein orifice openings by two guide wires. The first guide wire is pushed axially through the distal portion of the catheter, while the second guide wire inclines proximally from the ablation applicator through the catheter.

Substantial drawbacks of the prior art ablation device reside in that the active length of the ablation applicator is defined by the distance between the inlet for the first guide wire at the distal end and the position where the second guide wire, in a proximal position, passes through the catheter. In practice, the distance between the pulmonary vein orifice openings differs anatomically from one patient to the next so that a set of ablation catheters with ablation applicators of varying lengths will be required for treating various patients, disregarding the fact that the lesion is not optimally suited to the individual anatomy.

### SUMMARY OF THE INVENTION

It is an object of the invention to improve an ablation device of the type mentioned at the outset in such a way that it is flexibly and optimally suitable to varying anatomic conditions.

This object is attained by the invention according to which, in addition to the ablation catheter with the ablation applicator, provision is made for a steerable positioning catheter which the ablation catheter is guided along for displacement in the longitudinal axial direction. This guide terminates proximally before a preferably steerable distal end section on the positioning catheter so that the ablation catheter with the ablation applicator is freely movable in this area that stands out beyond the guide. This construction further enables the distal end of the positioning catheter to be led into the second associated vessel orifice over a certain distance. The effective ablation length, outside the guide, of the ablation applicator becomes variable by longitudinal axial displacement of the two catheters relative to each other.

The positioning catheter can be controllably moved into a desired position by the aid of a wire pull or a separate guide wire or catheter.

According to the invention, the ablation device can be optimally adapted to respective anatomical conditions by this variability of the effective ablation length.

Detailed information and further features and advantages of the invention will become apparent from the ensuing description of exemplary embodiments of the subject matter of the invention, taken in conjunction with the drawings.

### BRIEF DESCRIPTION OF THE DRAWINGS

FIG. 1 is a diagrammatic illustration of a first embodiment of an ablation device upon application of a lesion;

FIG. 2 is an illustration, by analogy to FIG. 1, of a second embodiment of an ablation device;

FIGS. 3A to C are side views of details of the two side by side catheters of the ablation device according to FIGS. 1 and 2; and

FIGS. 4A to C are sectional views of the two catheters along the lines IV-IV of FIGS. 3A to C.

### DESCRIPTION OF THE PREFERRED EMBODIMENTS

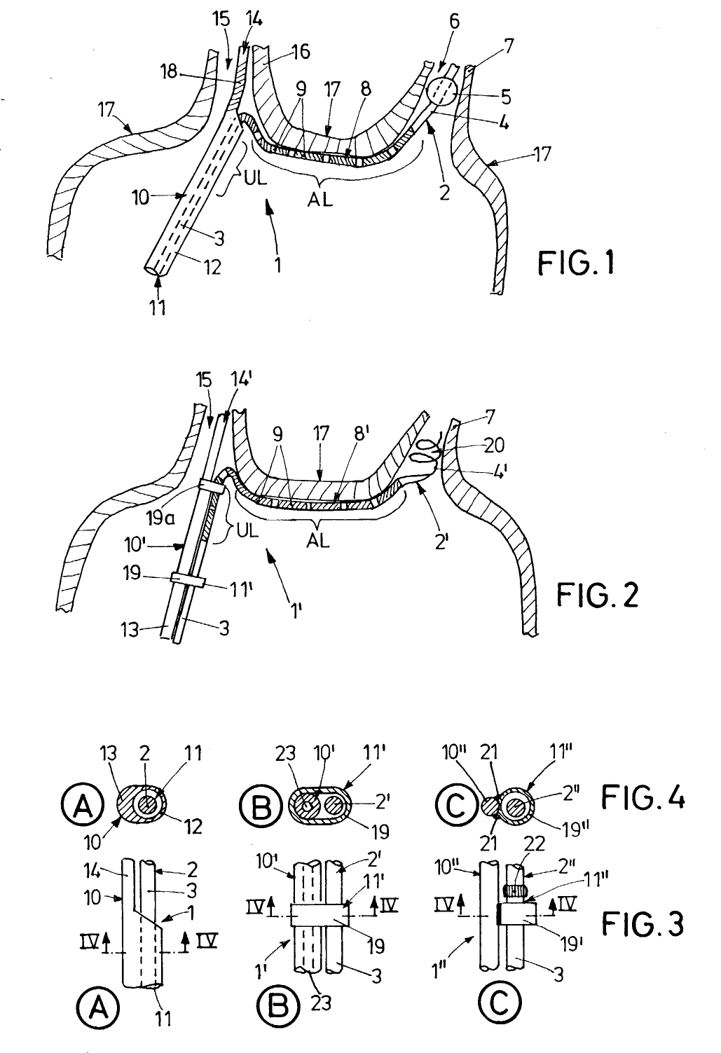

The fundamental structure of the ablation device 1 and the application thereof in the heart is going to be explained in conjunction with FIG. 1. Provision is made for a first catheter—the ablation catheter 2—which conventionally comprises a long thin shank 3 with a proximal end (not shown) and a distal end 4.

The distal end 4 of the ablation catheter 2 is equipped with a dilatable balloon 5 as an abutment, serving for fitting the catheter 2 in a body orifice such as an orifice opening 6 of a pulmonary vein 7 into the atrium of the heart.

Proximally before the balloon 5, the ablation catheter 2 is provided with an ablation applicator 8 over a length in the order of magnitude of 10 cm, the ablation applicator 8 comprising for example a number of aligned, highly flexibly ring electrodes 9. Via these ring electrodes 9, high frequency current can be emitted in a manner still to be explained, for a lesion to be produced.

As seen in FIG. 1, provision is made for a second catheter—the so-called positioning catheter 10—which, by analogy to the ablation catheter, may include a lumen for a guide wire or a control wire and is correspondingly controllable by way of a control and deflection mechanism. The positioning catheter 10 generally serves for guiding and positioning the ablation catheter 2. To this end, the positioning catheter 10 comprises a guide, designated by 11, for the ablation catheter 2, the guide 11 being formed by a lumen 12 inside the positioning catheter 10 in the exemplary embodiment of FIG. 1. As seen in FIGS. 3A and 4A, the positioning catheter has a rounded oblong cross-sectional shape along most of its length, with the lumen 12 being eccentrically integrated therein. At the distal end of the guide 11, this cross-sectionally rounded oblong shank 13 is continued by a distal end section 14 that is controllable by the means mentioned. The effective ablation length AL, outside the guide 11, is variable by longitudinal axial displacement of the two catheters 2, 10 relative to each other.

Applying a linear lesion between the mentioned orifice opening 6 of the pulmonary vein 7 and the adjacent orifice opening 15 of a second pulmonary vein 16 will be explained in conjunction with FIG. 1. First the ablation catheter 2 is led by its distal end 4 into the orifice opening 6 of the pulmonary vein 7 by being advanced towards it on the positioning catheter. Then the balloon 5 is being inflated for the distal end 4 to be dilated and fixed.

Then the positioning catheter 10 is retracted and manoeuvred towards the orifice opening 15 of the second pulmonary vein 16. In the process, the effective ablation length AL of the ablation catheter 12 is uncovered. The distal end section 14 of the positioning catheter 10 is pushed sufficiently far into the pulmonary vein 16 for the effective ablation length AL to rest stably on the tissue. The ineffective length UL of the applicator 8 is kept within the guide 11 by corresponding setting. Accurate adaptation of the ablation length AL to the respective anatomy will result in a proper lesion of precise length being produced by application of high frequency current via the ablation applicator 8. For excellent energy transfer from the applicator 8 to the myocardial wall 17, the positioning catheter 10 is pushed in a direction into the pulmonary vein 16 so that the ablation applicator 8 is mechanically pressed against the myocardial wall 17.

As roughly outlined by hatching in FIG. 1, the positioning catheter 10 may have an additional ablation applicator 18 in the vicinity of its distal end section 14.

In the embodiment, seen in FIG. 2, of the ablation device 1′, the guide 11′ of the ablation applicator 8′ on the positioning catheter 10′ differs from the design according to FIG. 1. The guide 11′ includes guide rings 19 which line up on the positioning catheter 10′ at a distance from each other. The last distal guide ring 19a marks the end of the guide 11′ which is topped by the non-guided distal end section 14′ of the positioning catheter 10′. As seen in FIG. 3B and 4B, the guide rings 19 are rounded oblong loops that are eccentrically tightly joined to the positioning catheter 10′. The free cross-sectional area of the loop guides the ablation catheter 2′. The embodiment according to FIGS. 3B and 4B further comprises a lumen 23, inside the positioning catheter 10′, for the guide wire mentioned at the outset.

Unlike the embodiment according to FIG. 1, the ablation catheter 2′ is provided with an expandable spiral tip 20, instead of a balloon, as fixing means of the distal end 4′. Shaping this spiral tip 20 may take place by a conventional wire pull or by it being made from memory metal.

Applying and, in particular, setting the effective ablation length AL for the ablation device 1′ of FIG. 2 does not differ from the ablation device 1 of FIG. 1.

In the embodiments of the ablation device 1″ seen in FIGS. 3C and 4C, the guide rings 19′ are not provided in the form of rounded oblong loops, as in the embodiment according to FIGS. 3B, 4B, but they are rings of a circular cross-sectional shape mounted on the positioning catheter 10′ by an appropriate joint 21 in the form of an adhesive or weld. The ablation catheter 2″ may be designed by analogy to FIGS. 1 or 2. As seen in FIG. 3C, in this embodiment by analogy to the design of FIGS. 2, 3B and 4B, the last guide ring 19′ marks the end of the guide 11″ and the beginning of the distal end section 14 of the positioning catheter 10″. As roughly outlined in FIG. 3C, the ablation catheter 2″, in front of its distal end, may be provided with a thickening 22 which prevents this catheter from slipping out of the guide 11″.

The length of the distal end sections 14, 14′ of the positioning catheters 10, 10′, 10″ may be in the range of between 5 and 50, preferably between 10 and 30 mm.

For the sake of completeness, the following technical details in the ablation devices 1, 1′, 1″ still remain to be mentioned, which are not explicitly shown in the drawings:

In addition to the way of fixing the ablation catheter 2, 2′ by means of a balloon 5 or a spiral tip 20, other fixing means in the form of for instance hooks or helices may be provided on the distal end of the catheters 2.

The ring electrodes 9 of the ablation applicators 8, 8′ may be made from for example flexible spiral metal or flexible conductive plastic material, ensuring as high a flexibility as possible.

The application fluid of the ablation applicator may approach inside the catheter and it may be transmitted to the tissue via a conductive fluid (for example salt solution). The fluid may be supplied through an additional rinsing lumen. Using a conductive fluid has the advantage of any blood in the vicinity of energy output being simultaneously washed away from the ablation position, which reduces the risk of coagulation. A combination of external electrodes and rinsing is conceivable.

Ablation being regularly accompanied with some thermal effect on the tissue of the heart, the energy-produced effect can be monitored by thermo-sensors in the catheters 2 and 10.

What is claimed is:

1. An ablation device for cardiac tissue, in particular for forming linear lesions between two vessel orifices (6, 15) in a heart, comprising
an ablation catheter (2, 2′, 2″) which, in front of a distal end, is provided with an ablation applicator (8, 8′) that reaches over a certain length of the catheter;
a steerable positioning catheter (10, 10′, 10″) which the ablation catheter (2, 2′, 2″) is guided along for displacement relative thereto in a longitudinal axial direction; and
a distal end section (14, 14′) of the positioning catheter (10, 10′, 10″), proximally of which a guide (11, 11′, 11″) of the ablation catheter (2, 2′, 2″) through the positioning catheter (10, 10′, 10″) terminates and the ablation catheter (2, 2′, 2″) is freely movable together with the ablation applicator (8, 8′), with an ablation length (AL), outside the guide (11, 11′, 11″), of the ablation applicator (8, 8′) being variable by longitudinal axial displacement of the ablation catheter (2, 2′, 2″) and the positioning catheter (10, 10′, 10″) relative to each other.

2. An ablation device according to claim 1, wherein the ablation catheter (2) is displaceably guided in a lumen (12) of the positioning catheter (10).

3. An ablation device according to claim 1, wherein the ablation catheter (2, 2″) is displaceably guided within an alignment of guide rings (19, 19′) which are spaced apart along the positioning catheter (10′, 10″), with a non-guided end section (14′) being located ahead of a distally last guide ring (19a).

4. An ablation device according to claim 1, wherein at least one of the ablation catheter (2, 2′, 2″) and positioning catheter (10,. 10′, 10″) is provided with a lumen (23) for a guide or control wire.

5. An ablation device according to claim 1, wherein the ablation catheter (2, 2′), at its distal end (4, 4′), is provided with fixing means (5, 20) for being tightly fitted in a cardiac vessel orifice (6).

6. An ablation device according to claim 5, wherein the fixing means include a deflatable catheter balloon (5).

7. An ablation device according to claim 5, wherein the fixing means include a distal spiral tip (20).

8. An ablation device according to claim 1, wherein the distal end section (14, 14′) of the positioning catheter (10, 10′) has a length of 5 to 50 mm, preferably 10 to 30 mm.

9. An ablation device according to claim 1, wherein the distal end section (14, 14′) of the positioning catheter (10, 10′) is provided with an additional ablation applicator (18).

10. An ablation device according to claim 1, wherein the distal end section (14, 14′) is independently controllable.
